(19) United States
(12) Patent Application Publication
(10) Pub. No.: US 2008/0015528 A1
(43) Pub. Date: Jan. 17, 2008

(54) Clothing article incorporated with urine drainage device

(76) Inventor: Kuang-Chih Chang, Nantou City (TW)

Correspondence Address: CHARLES E. BAXLEY, ESQ., 90 JOHN STREET, THIRD FLOOR, NEW YORK, NY, 10038, US

(21) Appl. No.: 11/485,672

(22) Filed: Jul. 13, 2006

(51) Int. Cl. A61F 5/44

(52) U.S. Cl. 604/353; 604/349

(57) ABSTRACT

An article of clothing incorporated with a urine drainage device according to the present invention primarily comprises: a clothing article having an opening at the front region sheet positioned corresponding to a wearer's urinary organ, a foundation settled about said opening having a round hole, a foundation with a round hole disposed in the opening, a drainage device which consists of a urinary-organ sleeve and a conduit wherein the urinary-organ sleeve has a joint at its one end detachablely attached to said foundation and the conduit may be extend to a urine receptacle. When a male patient wears the disclosed subject matter, his urinary organ may be accommodated in the urinary-organ sleeve so that the emitted urine can be led to the conduit through the conical funnel and then be collected in the mentioned urine receptacle.

### BACKGROUND OF THE INVENTION

1. Technical Field

The present invention is related to an object used for nursing incontinence patients and more particularly to an article of clothing incorporated with urine drainage device.

2. Description of Related Art

Fecal incontinence may be one of the commonest problems to paralyzed and long term bed-ridden patients and the currently typical approach for this problem is using disposable diapers. Despite today's diaper products have been improved on both absorptivity and air permeability, infections of skin or urinary system can not be precluded completely. The main reason of the problems is that a care-giver can hardly check the patients' fecal states at short intervals and it results the fecal-absorbed diaper remains an undesirably damp, muggy and stuffy environment till the care-giver replaces it. During this waiting term, germs may generate in a significant way and therefore incur infections of the patients' skins or urinary systems.

Though it may be a solution to ask a care-giver to observe the patient from time to time for changing the diapers timely, however, it is not easy to be achieved especially in hospitals or nursing homes where each care-giver may be charged with the care of plural patients. Additionally, it may cause detrimental disturbance to the patients to frequently uncover the quilts and turn the patients over for checking the diapers.

### SUMMARY OF THE INVENTION

The present invention has been accomplished under the circumstances in view. The invention provides a clothing article incorporated with a urine drainage device as a solution to foresaid problems. As a patient wears the disclosed subject matter, the emitted urine thereof can be collected by a drainage device and then led to a receptacle.

The article of clothing incorporated with urine drainage device according to the present invention primarily comprises: an clothing article having an opening at the position corresponding to a wearer's urinary organ, a foundation positioned about said opening having a round hole for allowing the wearer's urinary organ passing through, a drainage device detachablely attached to said foundation at its one end further with the other end connected with a conduit which may be extend toward a urine receptacle.

It is one object of the present invention to provide a clothing article incorporated with a urine drainage device, which facilitates preventing the clothing article from getting feculent and keeping it dry and clean by collecting and draining the wearer's urine with a urine drainage device. By this way, the opportunity that the wearer gets skin infection or urinary system infection by the reason of fecal matter can be significantly reduced.

It is another object of the present invention to provide a clothing article incorporated with a urine drainage device, which releases the care-giver from burden of frequent check of the patients. It also allows the care-giver simply observing the patients' urinary states through the claimed drainage device without disturbing the patients recurrently.

It is still another object of the present invention to provide a clothing article where a urine drainage device can be easily assembled or disassembled with through the foundation that permits the care-giver cleaning or replacing the drainage device and foundation conveniently.

### BRIEF DESCRIPTION OF THE DRAWINGS

FIG. 1 is an exploded view of the urine drainage device applied to an article of clothing according to the present invention.

FIG. 2 is a perspective view showing the urine drainage device combined with an article of clothing according to the present invention.

FIG. 3 is a sectional view taken along Line 3-3 of FIG. 2.

FIG. 4 is an exploded view depicted according to the profile shown in FIG. 3.

### DETAILED DESCRIPTION OF THE PREFERRED EMBODIMENT

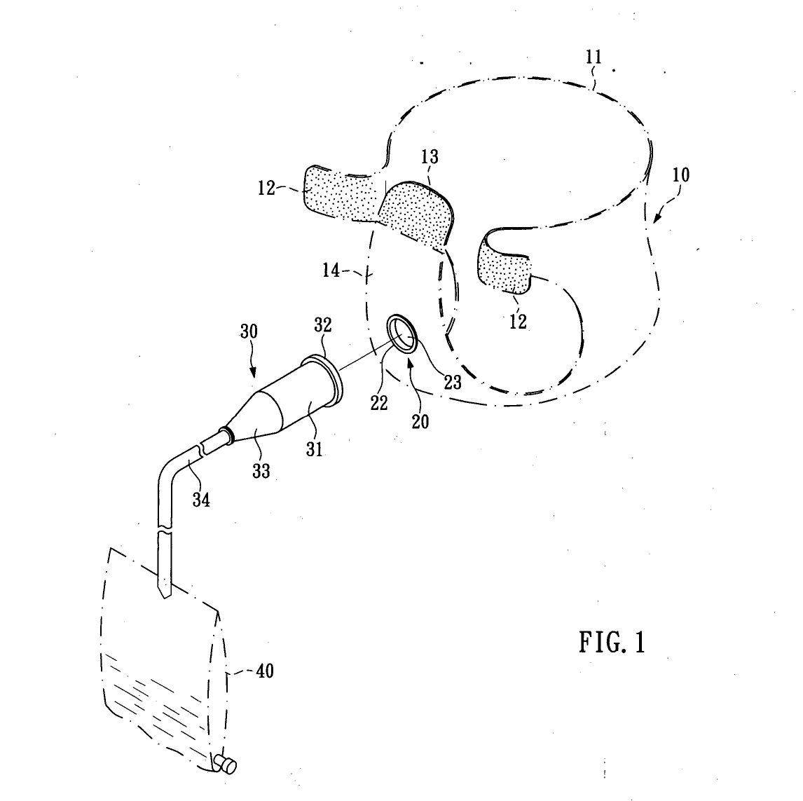

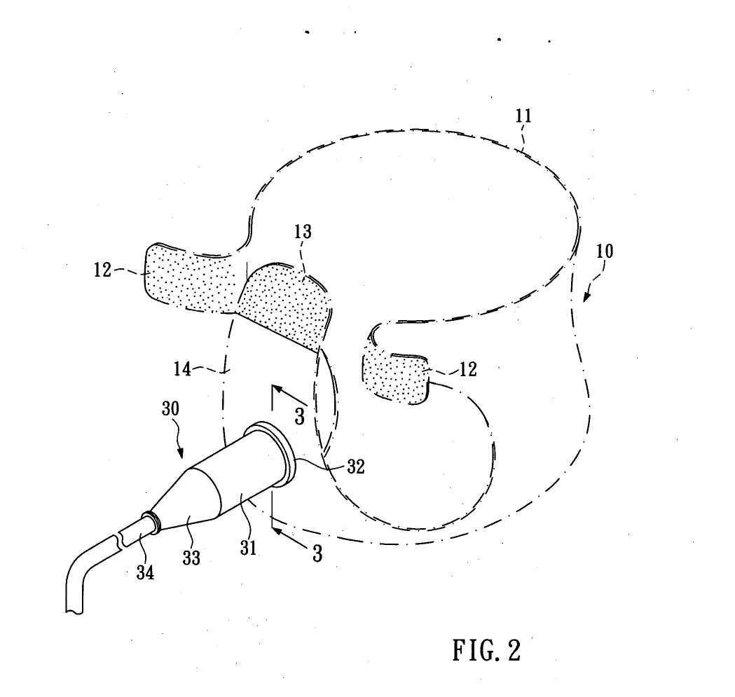

The preferred embodiment expressing the primary technique of the present invention is shown in FIGS. 1 and 2 which are depicting the urine drainage device applied to a clothing article. This embodiment is particularly designed for a male wearer. As seen in the FIGS., the clothing article 10 may be a disposable diaper or the like. There are two tape fasteners 12 extended from each side of the rear waist region 11, which are releasably fastened to the tape fastener 13 provided on the front waist region for fixing the clothing article on the wearer's body, a foundation 20 positioned on the clothing article 10, a drainage device 30 which is attachable to the foundation 20 further comprising a urinary-organ sleeve 31 whose proximal end is connected with a joint 32 and the other end is associated to a conical funnel 33; and a conduit 34 extended from the conical funnel 33. When wearing such a clothing article, a male wearer may dispose his urinary organ in the urinary-organ sleeve 31 so that the emitted urine can be led to the conduit 34 through the conical funnel 33 and then be collected in the mentioned urine receptacle 40. As the embodiment shown in FIG. 1, the urine receptacle 40 can be a urine bag.

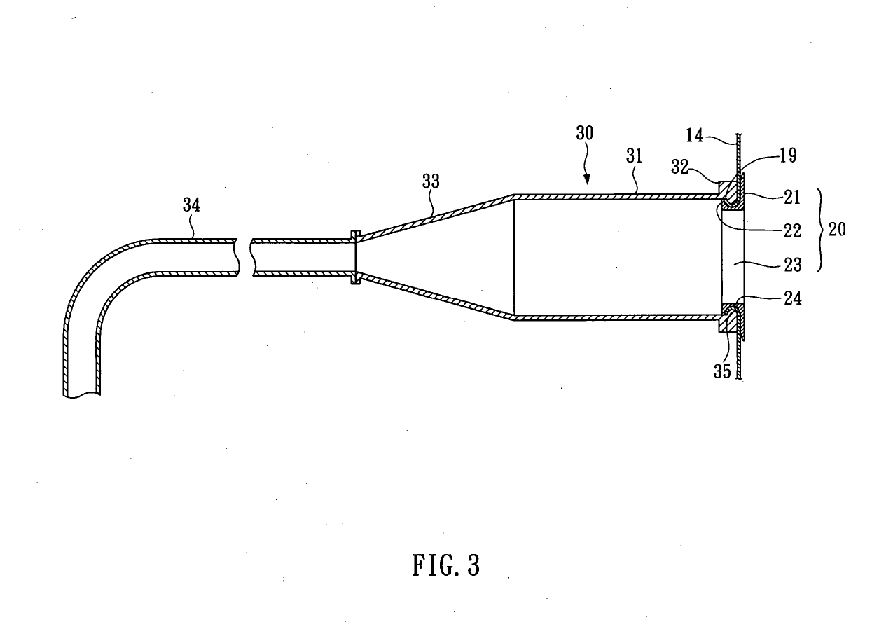

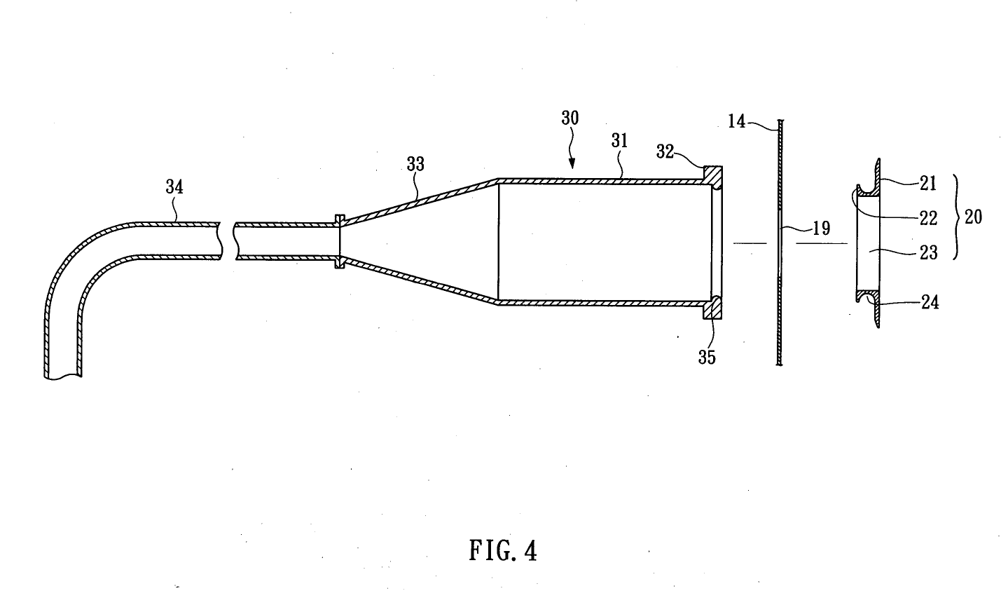

FIG. 3 provides a partial sectional view taken along Line 3-3 of FIG. 2 and FIG. 4 is an exploded drawing depicted according to the profile shown in FIG. 3. As can be seen in the figures, the foundation 20 consists of a base plate 21 and a ring 22 which are integrately formed. A round hole 23 is provided on the base plate 21 at the position corresponding to the ring 22. An engagement device 24 is provided at the external periphery of the ring 22 for getting engaged with the joint 32 of drainage device 30. In the embodiment illustrated by the figures, the engagement device 24 is an annular groove. The foundation 20 is fixed at the inside surface of the front sheet 14 of the clothing article by the base plate 21 in the way that the central region of the ring 22 is aligned with the opening 19 of the clothing 10. A flange 35 arranged at the joint 32 of the drainage device 30 can be engaged with the annular groove 24 of said foundation 20 to clench the peripheral edge of the opening 19 of the clothing article 10 thereinbetween so that the foundation 20 and drainage device 30 can be fixedly fastened on the clothing article 10. The conduit 34 is a flexible hose which is sealedly combined with the distal end of the conical funnel 33 by means of any available known technique such as ultrasonic bonding, agglutination or fusion splicing that is not limited herein.

Although a particular embodiment of the invention has been described in detail for purposes of illustration, it will be understood by one of ordinary skill in the art that numerous variations will be possible to the disclosed embodiments without going outside the scope of the invention as disclosed in the claims.

What the invention claimed is:

1. An article of clothing incorporated with a urine drainage device primarily comprises:
an article of clothing having an opening provided on a front region sheet thereof at the position corresponding to a wearer's urinary organ,
a foundation positioned about said opening having a round hole;
a cannular-shaped drainage device detachably attached to said foundation at one end thereof with the other end associated to a conduit.

2. The article of clothing incorporated with a urine drainage device as claimed in claim 1, wherein the foundation includes a base plate having a ring disposed thereon and the round hole thereof is positioned corresponding to the central region of the ring, and wherein, the ring is settled in the opening of the front region sheet and the base plate is arranged at the inside surface of front region sheet.

3. The article of clothing incorporated with a urine drainage device as claimed in claim 2, wherein the urine drainage device includes a urinary-organ sleeve which has a joint at one end thereof and is connected to the conduit at the other end thereof.

4. The article of clothing incorporated with a urine drainage device as claimed in claim 3, wherein an engagement device is provided at the external periphery of the ring to engage with the joint of the drainage device.

5. The article of clothing incorporated with a urine drainage device as claimed in claim 4, wherein the engagement device is an annular groove and a flange is provided at the joint of the drainage device to be engaged with the annular groove.

6. The article of clothing incorporated with a urine drainage device as claimed in claim 5, wherein the edge of the opening of the clothing is clenched between said flange and engagement device.

7. The article of clothing incorporated with urine drainage device as claimed in claim 1, wherein the distal end of the conduit is associated to a urine receptacle.
